(19) United States
(12) Patent Application Publication
(10) Pub. No.: US 2021/0369767 A1
(43) Pub. Date: Dec. 2, 2021

(54) Method of treating and/or preventing viral infections

(71) Applicants: Vladimir Oks, (US); Andrey Oks, (US)

(72) Inventors: Vladimir Oks, Medford, MA (US); Andrey Oks, Freehold, NJ (US)

(21) Appl. No.: 17/403,813

(22) Filed: Aug. 16, 2021

Related U.S. Application Data
(63) Continuation-in-part of application No. 16/825,441, filed on May 7, 2020.

(51) Int. Cl. A61K 33/00; A61P 31/16; A61P 31/14

(52) U.S. Cl. CPC A61K 33/00; A61P 31/14; A61P 31/16

(57) ABSTRACT

A method of treating and/or preventing viral infections caused by common cold, influenza, and coronaviruses with or without symptoms is based on a fact that virus's survival time depends on the temperature and relative humidity, and an assumption based on scientific tests that a virus can be inactivated/killed or its ability to penetrate and replicate copies inside living cells can be weakened in high temperature environment by heat exposure to the virus. Creating high temperature environment in the upper respiratory and gastrointestinal (GI) tracts and preventing spread of the virus into the lower part of the respiratory tract using continuous and prolonged hot water exposure on the virus prevents development of pneumonia and expedite recovery.

### FIELD OF THE INVENTION

The present invention relates to the treatment of a viral respiratory infectious diseases by heat therapy. The objective of the present invention is to destroy viruses by heat in the upper respiratory and gastrointestinal (GI) tracts and prevent the development of pneumonia and other complications. Study of this method is based on a human body temperature regulation model, represented by a feedback control system under the influence of viruses and the effect of the present method.

### BACKGROUND OF THE INVENTION

### Common cold

The common cold is a very frequent acute illness in industrialized societies and the leading cause of visits to the physicians in the United States. Though it is usually benign, it is a leading cause of absence from work, also causing a significant economic burden including loss of productivity and treatment costs. The common cold is caused by a variety of viruses, most of the time with rhinoviruses, coronaviruses, adenoviruses, and enteroviruses. There is currently no cure for common cold, so that current therapy targets symptoms relief. Prevention strategies for the common cold include avoiding infected people and frequent hand washing during cold season.

There are various applications of heat in the treatment of colds. Certain so-called face saunas, for the isolated treatment of the head region, were developed for treating colds or dry saunas have been recommended by so-called holistic therapists. From U.S. Pat. No. 7,100,605 a device is known which is constructed in the form of a hand-held hair dryer for the localized hypothermic treatment of colds whereby an ionized heated current of air which is warmed up and acts directly via the nose containing crystallized salt on which a current of warm air impinges. This therapies device as well as most inhalation devices is able to cause the symptoms of cold to relief, but they nevertheless have a few serious disadvantages. The main disadvantage for existing methods and devices that use heat exposure on the virus, and for this device particularly is that exposure on the virus implemented with the relatively low temperature and short period of time: “The whole process of inhaling for 3 minutes of air through the nose at a temperature of 37° C. to 54° C., preferably of 41° C. to 44° C.” even if it “should be repeated five times” with intervals 1-1.5 hour. Existing methods or devices with external heat exposure to the body (face saunas, dry saunas, hot bath, sun, hot air, UV) cannot effect a cure of infection because the external heat does not increase enough the upper respiratory and GI tracts temperature, does not change the inner body, and the human central body temperature to inactivate/kill a virus but can only relief the symptoms. They can relief symptoms “but will not make your cold go away any faster” (CDC Features, Common Colds: Protect Yourself and Others) as well as steam inhalation can afford some relief but never effect a cure.

Conventional therapies have a limited efficacy. Certain drugs are costly and have side effects. Dietary supplements (e.g., Echinacea, ginseng, etc.) are often believed to be effective, but they lack formal studies using modern culture methods.

### Flu

Flu is a contagious illness caused by the influenza virus. Common flu is characterized by an acute infection associated with respiratory problems, intense muscle pain, headaches, chills, nasal obstruction, fever, cough, sneezing and sore throat. The majority of the population will generally recover from flu without any complication. Older people, young children and people with deficient immune systems, may have serious complications or may even die from the flu.

Current literature describes human common influenza as a serious disease causing each year an estimated 36,000 deaths in the United States only. Yearly common influenza (flu) epidemics result in lost workdays and schooldays as well as a significant number of hospitalization days and even deaths among the elderly, seniors being particularly vulnerable to respiratory infections.

New influenza strains infecting humans therefore appear from time to time. These strains frequently originate from other species and have adapted to human through mutations. The swine influenza A H1N1 flu virus, also named Swine Flu, has been reported around the world and was declared a pandemic influenza virus. Swine flu H1N1 originally only affected pigs but started infecting humans in North America in 2009. Humans having little to no natural immunity to this virus, it can lead to a serious and widespread illness.

Vaccines can prevent flu in 70 to 90% of the cases observed in healthy adults. However, the flu vaccine has to be redesigned each year because the previous year's vaccines are likely to be ineffective against the newly mutated strains. The yearly vaccine is therefore designed before the season begins based on that from the previous year. Since scientists usually cannot predict with accuracy which strain will be dominant, the vaccine may not fully correspond to the new strain. In addition, the flu vaccine is specifically designed against influenza virus strains A and B and does not therefore protect against cold viruses such as rhinovirus and corona types.

Flu symptoms can be alleviated with either the previously mentioned treatment or with, for example, 1) over-the-counter analgesics to relieve pain and reduce fever; 2) cough suppressants for dry cough with no mucus; 3) expectorants to help clear mucus so it can be coughed up; and/or 4) decongestants to reduce nasal congestion.

### Coronaviruses

Coronaviruses vary significantly in risk factor. Some can kill more than 30% of those infected and some are relatively harmless, such as the common cold. Coronaviruses cause colds with major symptoms, such as fever, and sore throat from swollen adenoids, occurring primarily in the winter and early spring seasons. Severe acute respiratory syndrome coronaviruses like SARS-CoV, MERS, COVID-19 can cause pneumonia (either direct viral pneumonia or a secondary bacterial pneumonia) and bronchitis (either direct viral bronchitis or a secondary bacterial bronchitis) in any season during year and in any climate. The much publicized human coronavirus discovered in 2003, SARS-CoV, which causes severe acute respiratory syndrome (SARS), has a unique pathogenesis because it causes both upper and lower respiratory tract infections.

Many scientific publications provide data on the dependence of virus survival time on temperature and humidity on surfaces of different materials.

For example, coronavirus Survival Times at Relative Humidity & Air Temperatures in Hours & Days: (K. H. Chan, 2011; Smith R D, 2006)
The survival times at 80% RH and 40° C. were less than 7 hours for proxies of coronaviruses on stainless steel.*
The survival times at 50% RH and 40° C. were more than 24 hours.* There was a reduction in the ratio of virus to −3 Log10 (Nt/NO) in 24 hours.
The survival times at 20% RH and 40° C. were more than 120 hours.* There was a reduction in the ratio of virus to −3 Log10 (Nt/NO) in 120 hours.
The survival times at 50% RH and 20° C. were less than 7 days on stainless steel.*
For proxies of coronaviruses on stainless steel. The proxies used were transmissible gastroenteritis virus (TGEV) and mouse hepatitis virus (MHV).

Some scientific publications showed that heat treatment of SARS-CoV for 45 min at 75° C. resulted in inactivation of the virus, while 90 min at 56 and 65° C. was required for virus inactivation (Darnell, M. E. R., et al., Inactivation of the coronavirus that induces severe acute respiratory syndrome, SARS-CoV, 2004). This publication states that “data are similar to the findings of Duan et al. (2003), wherein viral inactivation occurred at 90, 60, and 30 min after incubation at 56, 65, and 75° C., respectively. Heat is an effective means of SARS-CoV inactivation; however, stocks containing viral aggregates may require a longer duration of heat exposure”. While no studies have yet been published on the virus that causes Covid-19 and its resistance to high temperatures, it is likely that it will be similar to other coronaviruses.

Different scientific papers have different data, but the coronavirus's survival time tendency to depend on temperature and relative humidity continues.

Viruses spread from person to person primarily via respiratory droplets from coughing or sneezing. People are thought to be most contagious when symptomatic, though some spread might be possible before symptoms show. The time between exposure and symptom onset is typically five days, but may range from two to fourteen days. Common symptoms include fever, dry cough and shortness of breath. Complications may include pneumonia and acute respiratory distress syndrome. There is no specific antiviral treatment, except vaccines that are likely to be ineffective against the newly mutated strains, but research is ongoing. Efforts are aimed at managing symptoms and supportive therapy. Recommended preventive measures include handwashing, wearing facemask, using hand sanitizer, maintaining distance from other people (particularly those who are unwell), and monitoring and self-isolation for fourteen days for people who suspect they are infected.

Public health responses have included national pandemic preparedness and response plans, travel restrictions, quarantines, curfews, event postponements and cancellations, and facility closures. These include a quarantine of Hubei, China; the nationwide quarantines of Italy, Spain, the Czech Republic, and Germany; curfew measures in China and South Korea; various border closures or incoming passenger restrictions screening methods at airports and train stations; and travel advisories regarding regions with community spread.

Symptoms of COVID-19 are non-specific and those infected may either be asymptomatic or develop flu symptoms such as fever, cough, fatigue, shortness of breath, or muscle pain. Further development can lead to severe pneumonia, acute respiratory distress syndrome, sepsis, septic shock and death. Some of those infected may be asymptomatic, returning test results that confirm infection but show no clinical symptoms, so researchers have issued advice that those with close contact to confirmed infected people should be closely monitored and examined to rule out infection.

### THE MAIN SCIENTIFIC SUBJECTS, ON WHICH THE PRESENT METHOD IS BASED

This method is based on the theory of anthropology science, studying the process of historical and evolutionary formation of humans. The method is a natural extension of human evolutionary development, since the human use of fire, in the fight for its existence, in the fight against foreign bodies in an organism including in the fight against viruses.

This method is subject to the science of biology, the natural science that studies life and living organisms especially its immunology branch that covers the study of immune systems in all organisms by the physiological functioning of the immune system in both state of health and state of illness, and the body's response to foreign bodies. The main purpose of the immune system is to protect against intrusions and to preserve the integrity of the internal environment of the body, its biological individuality.

This method is subject to the laws of cybernetics, the science of general patterns of information management and transmission processes in machines, living organisms and society. Cybernetics includes the study and impact of feedback, on management and communication in living organisms, machines and organizations, including self-organization, and regulation of all processes in nature including human activity and society.

This method is subject to the laws of mechanics—a section of physics, science that studies the movement of material bodies and the interaction between them. An individual is the most complex object not only on Earth, but perhaps in the entire universe. Taking into account the evolutionary development of man and progress of modern technologies, man can be considered as a material, mechanical object, at least its physical body's parts and systems describe by physics, physiology, biology and microbiology using simplified description of any human function by a small number of mathematical equations.

This method is subject to the laws of energy—a section of physics, science that studies the quantitative property that must be transferred to an object in order to perform work on, or to heat, the object.

Energy is a conserved quantity; the law of conservation of energy states that energy can be converted in form, but not created or destroyed. Common forms of energy include the chemical energy released when a fuel burns and the particularly thermodynamic heat energy transfer due to an object's temperature.

This method is subject to the science of chemistry—the discipline, which is involved with the structure, properties and behavior of elements and compounds and the changes they undergo during a reaction with other substances, specifically its sub discipline of biochemistry's field of metabolism, the set of life-sustaining chemical reactions in organisms.

This method is subject to the laws of the continuous-time world. Its main property is the continuity of the time variable, i.e. there is a finite value of the any process's function in any moment of the time. The functioning of all phenomena in nature, including human nature, is carried out in an integral dependence on changes under the influence of internal and external impacts.

### SUMMARY OF THE INVENTION

Goals of the present method of treating and/or preventing viral infection are to:
a. inactivate/kill a virus in the upper respiratory and GI tracts,
b. prevent complications, especially pneumonia,
c. expedite recovery,
d. prevent viral diseases.

A method of treating and/or preventing viral infections caused by common cold, influenza, and coronaviruses with or without symptoms is based on a fact that virus's survival time depends on the temperature and relative humidity, and an assumption based on scientific tests that a virus can be inactivated/killed or its ability to penetrate and replicate copies inside living cells can be weakened, by heat exposure to the virus. Creating such high temperature environment in the upper respiratory and GI tracts and preventing spread of the virus into the lower part of the respiratory tract using continuous and prolonged heat exposure to the virus, prevents development of pneumonia and expedite recovery.

It is known that the survival time of the virus depends on the temperature: the higher temperature and the longer the heat exposure to the virus, the greater the possibility of inactivation of the virus. The present invention is designed to create a high-temperature environment in the upper respiratory and GI tracts where respiratory viral infection is mainly concentrated. With the present method, an agent delivering heat is hot, potable, boiled water the simplest, cheapest and natural product for a human. It is the most acceptable agent for the human body that can create such conditions for the environment in the upper respiratory and GI tracts. The consumption of hot water for a long period of time with high temperature inactivates/kills viral infection. Hot water delivers much more oxygen to the body, improves blood circulation, improves metabolism, reduces painful contractions of muscles, relieves nasal congestion, aids digestion, calms central nervous system, helps relief constipation, helps reduce toxins, and keeps you hydrated.

The method further reduces the risk of pneumonia by washing viruses away from the respiratory tract to GI tract. At the same time the hot water prevents development of a viral infection in the GI and urinary tracts. The present method prevents or reduces the spread of a virus into the lower respiratory tract and prevents development of pneumonia most effectively at the early stages of an illness, when an infection just starts to develop in the upper respiratory and GI tracts. A virus can be killed or its ability to penetrate and replicate copies inside living cells can be weakened, using continuous and prolonged hot water exposure on the virus.

Present method works similar to existing methods of application for medications. Traditional medications typically utilize a particular chemical compound or composition to kill a virus, or boost resistance to a virus, or create an immune system antibody. There are common rules for the use of any medication. For example—you caught a virus. Your personal, unique reaction to this virus, for this occurrence is a fever of 38.9° C. To manage your fever, you may take a fever-reducing medication. Your goal is to manage and return the temperature to normal human temperature. What medication, how many pills, what dose, how long, how frequent to take fever-reducing medication to bring the temperature to individual's normal range is unique to each user. The present method is unique and is based on a completely different approach, but the same rules apply for the present method within specified parameters:
apply this method as soon as you start to experience symptoms,
drink water at the personal maximal temperature that you can drink hot water,
drink continuously, with selected consumption rate, without stops or breaks,
drink for prolonged period of time with some, specified limitations, and
beyond specified limitations, drink until your symptoms have disappeared or have reduced and you feel better based on your subjective evaluation.

The present method utilizes temperature and time to kill or weaken any respiratory virus. If any respiratory virus is exposed to boiling water, it will be killed or inactivated in a matter of seconds with timing different for common cold, influenza, coronaviruses like SARS, MERS, COVID-19, and other respiratory viruses. Each viral infection corresponds to unique virus, responsive to unique therapies for each person or group of patients. Every individual should drinks hot water by present method according to person's unique physical constitution, biological, genetic, health conditions: metabolism, immune system, kidney, liver, heart, weight, and so on; type of virus, strain, stage of viral infection, symptoms, year's season, climate, population uniqueness. For example: in China the average weight of population is less than the average weight of population in the United States.

Water is a common fluid used for heat exchange, due to its availability and high heat capacity. It is especially effective to transport heat that will flow from the hot environment to the cold one in an attempt to equalize the temperature difference. This method works similar to the mechanism of heat transfer in a heat exchanger, where thermodynamic entropy for isolated system can be calculated. For some period of time of treating, the upper respiratory and GI tracts temperature becomes higher than internal body temperature because our body is not uniform in volume, mass, density from our head to our legs and energy from hot water disperses from point of injection throughout the body. Over time the temperature of the consumed water and the internal body temperature become equal. In other words, the entropy of the water has decreased as some of its energy has been transferred to the human body, of which the entropy has increased. The efficiency of application of hot water as a heat exchanger agent for heat exposure on the virus is much higher than the efficiency of the warm airflow supply used for inhalation. This method will require a lot of scientific research, which, taking into account previous works, to define all aspects of influence and interactions between human, virus, environment, hot water, temperature, heat exchange, time, location and so on that is taking into account all physical, physiological, biological, microbiological, chemical parameters of each substances. After detailed research, each country can create a list of applications instructions for each grouping of persons on how to use this method, similar to the use of existing medications.

### OBJECTIVES OF THE PRESENT METHOD

It is an objective of the present invention to create high temperature environment in the upper respiratory and GI tracts to prevent spread of the virus into the lower part of the respiratory tract and prevent development of pneumonia.

It is an objective of the present invention to expedite recovery.

It is an objective of the present invention to use this method as universal that can be utilized against viruses mentioned above, whose survival time depends on temperature.

It is an objective of the present invention to provide easily administered preparations that can be used at home by millions of people without medication or special equipment.

It is another objective of the present invention to use this method as a preventive measure. It is an additional proactive preventive measure, similar to a recommendation of “washing your hands”.

A further objective of the present invention is to provide a safe method with no side effects.

It is additional objective of the present invention to use hot, potable, boiled water the simplest, cheapest and natural product for a human, as an agent delivering heat.

A still further objective of the present invention is to provide preparations that are easy to administer and can be carried out and used anywhere so the user can continue the therapies throughout the day, days, weeks, and months.

It is another objective of the present invention to reduce a number of frequent acute illnesses causing a significant economic burden including loss of productivity, treatment costs, and to help slow down the spread of the cold, flu, and coronaviruses epidemics or pandemics.

It is also an objective of the present invention to treat and prevent viral infection and to help inactivate/kill other foreign bodies inside the GI and urinary tracts.

It is another objective of this invention to open a new chapter in research of hyperthermia application of this method for treatment other foreign bodies inside human organism including some types of herpes simplex or cancer cells and creation of special medical devices for treatment and prevention procedures.

### BRIEF DESCRIPTION OF THE DRAWINGS

This and/or other aspects and advantages of the present invention will become apparent from the following description of preferred embodiment, taken in conjunction with the appended drawings, in which:

FIG. 1 illustrates simplified model of a human body temperature regulation system, similar to a block diagram of feedback control system with a conventional integral controller.

FIG. 2 illustrates simplified model of a human body temperature regulation system under influence of a virus as a foreign body, introduced into a human organism, similar to a block diagram of feedback control system with a conventional integral controller and external source of disturbance.

FIG. 3 illustrates simplified model of a human body temperature regulation system under influence of a virus as a foreign body, into a human organism and usage of the present invention, similar to a block diagram of feedback control system with a conventional integral controller, external source of disturbance and disturbance compensator.

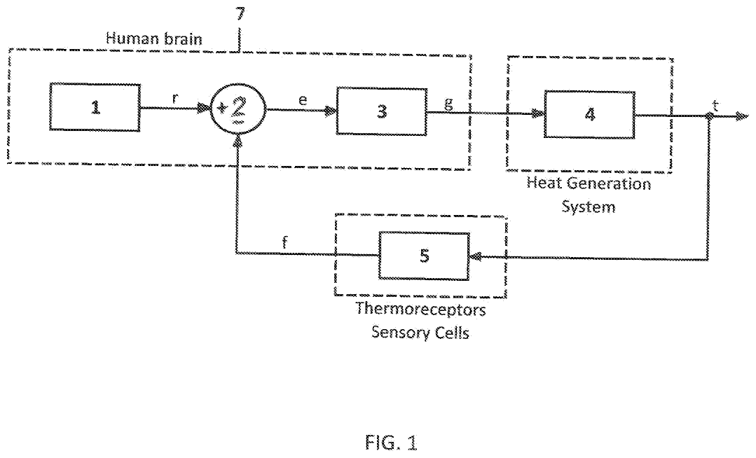

### DESCRIPTION OF THE FIG. 1

### Block Diagram of Feedback Control System

In FIG. 1 a set point unit 1 generates an input reference signal r, which is compared with the feedback signal f at the negative input of the summing unit 2, which in this case computes the difference between the signals, to obtain the deviation signal e. The deviation signal e is inputted into the integral controller 3 having transfer function Ki/s. The controller 3 generates, correspondingly, the integral of the deviation signal e multiply by Ki, variable signal g. The signal g is inputted into the controlled system block 4. The block 4 generates the output variable function t that is inputted into the feedback sensor block 5. The feedback sensor output signal f is entered into the negative input of the summing unit 2.

It is known that the transfer function of the integral controller is Gc(s)=Ki/s. Where: s—a Laplace operator; Ki—a gain, constant construction parameter of integral controller 3 and Ki=1/Ti; Ti—a time constant of integration of integral controller 3.

### Model of a Human Body Temperature Regulation

FIG. 1 also illustrates a simplified model of a human body temperature regulation system.

The Human Brain block 7 is an organ that serves as the center of the nervous system. Brain exerts centralized control over a body's nervous system and other organs by generating patterns for body activity. This centralized control allows rapid and coordinated responses to changes of internal and external conditions. Sophisticated, purposeful control of behavior based on a complex sensory input requires information integrating capabilities of a centralized brain.

The Human Brain block 7 includes a set point unit 1, the comparison device 2 and the regulator 3 that are parts of the regulation center of human body temperature—the Hypothalamus. The core temperature of a human is regulated and stabilized primarily by the hypothalamus, a region of the brain linking the endocrine system to the nervous system. As core temperature varies from the homeostatic set point, endocrine production initiates control mechanisms as part of homeostasis process to increase or decrease energy production or dissipation as needed to return the temperature toward the set point. The core temperature, which in the present invention is defined as the body temperature, most accurately is determined through rectal measurements. In this method, the temperature is measured in the mouth (oral). The rectal temperature is expected to be approximately 0.55 Celsius (or 1 Fahrenheit) degree higher than an oral temperature taken on the same person at the same time at stable conditions but not during application of present method.

Block 5—regular Thermoreceptors Sensory Cells, where free nerve endings or neurons that reside mostly in the skin, viscera, liver, skeletal muscles, brain, spinal cord transduce sensory feedback information f to the central nervous system in the brain block 2 in the hypothalamus. This feedback signal f is entered into the Brain block 2's input, with the opposite sign to the reference signal r, as negative feedback, to stabilize set body temperature t. The computed difference e between the signals is inputted into the integral regulator 3, which is also a part of the center of the nervous system Human Brain block 7.

The regulator's output signal g changes gradually and continuously. The output signal g change rate is determined by a parameter called the time constant of integration Ti and for each person is its own internal constant of temperature regulation. This signal g is inputted into the Heat Generation System of man's body, block 4 that maintains or changes body temperature. In thermoregulation of warm-blooded organisms, body heat is generated mostly in the deep organs, especially the liver, heart, in contraction of skeletal muscles, by vasoconstriction, blood flow, shivering, sweating, vasodilation, spinal cord, endocrine system, hormones, immune system, metabolism, endothermy, and homeothermy mechanisms of the temperature regulation of human body.

For example, the set point block 1 of the Human Brain 7 instructs to set body temperature at 36.7° C. The feedback sensor, Thermoreceptors Sensory Cells block 5 that measure temperature t of the body generates feedback signal f that is compared in the device 2, of the Human Brain's block 7 with an opposite sign to the reference signal r. Because signal f equal to signal r, the deviation signal e of the block 2 equal to zero. There is no change of signal g into Heat Generation System of man's body block 4. The Heat Generation System maintain body temperature, that corresponds to the specified by set point of Human Brain 36.7° C. In reality, the temperature cannot be kept perfectly at 36.7° C., as the process is integral, constantly and continuously changing, in the form of a saw, within man's body sensitivity, for example between 36.71° C. and 36.69° C.

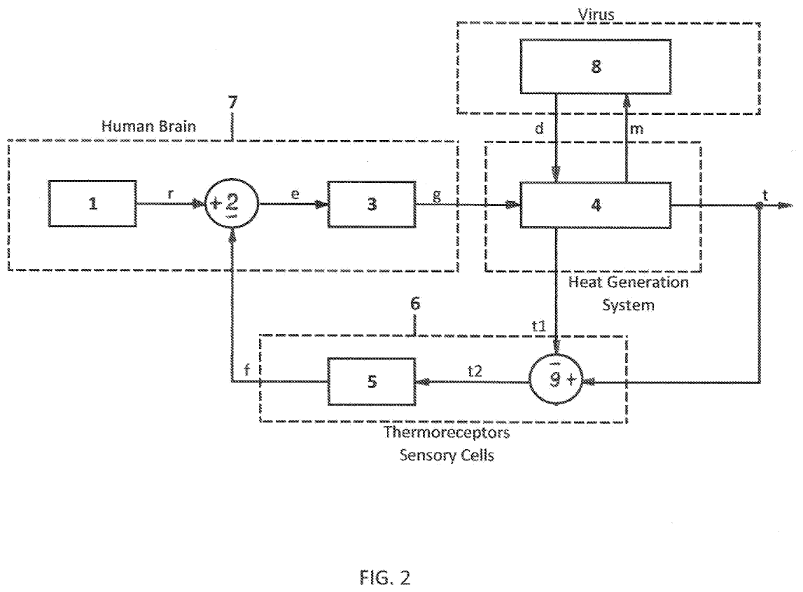

### DESCRIPTION OF THE FIG. 2

FIG. 2 is illustrated by FIG. 1 and presence of foreign bodies - viruses introduced into a human organism, to produce the same effect as an external source of disturbance into a block diagram of feedback control system with a conventional integral controller, where the external source of disturbance block 8 generates a disturbance signal d that is inputted into the controlled system block 4.

### Model of a Human Body Temperature Regulation with a Virus Disturbance

FIG. 2 illustrates the model of human body temperature regulation system under influence of a virus as a foreign body, where Virus is a source of disturbance block 8 and directly effects Heat Generation System block 4 by signal d. Concurrently, Heat Generation System block 4 directly responds to the Virus, the source of disturbance block 8 as a reaction to the virus, by signal m.

The Heat Generation System block 4 under influence of Virus signal d generates temperature disturbance signal t1 through feedback control mechanisms that are brought about by a natural resistance to change of internal, physical, and chemical functions maintained by living system. Signal t1 inputs into the summing unit 9—disturbance part of the Thermoreceptors Sensory Cells block 6. The output signal t2 of the summing unit 9 inputs into block 5, the regular part of the Thermoreceptors Sensory Cells block 6. This signal t2 is a combined signal of the temperature signal t from the positive input and the temperature disturbance signal t1 from the negative input of the summing unit 9.

At the beginning, the Virus block 8 disturbs body temperature balance by signal d, disrupting operations of certain mechanisms of the system generating heat in the human body and creating a favorable environment for itself. On the other hand, the Heat Generation System, block 4 converts, according to the law of energy conservation, and transfers part of the heat energy to counteract and fight viruses by signal m, influencing Virus block 8. The Heat Generation System under the influence of Virus signal d generates temperature disturbance signal t1. Within block 6, the summing unit 9 computes the difference between signal t from the positive input and signal t1 from the negative input, generating output signal t2 entering block 5, regular part of the Thermoreceptors Sensory Cells.

Under the influence of the Virus's disturbance source, the summing temperature signal t2 is decreased. The feedback signal f of the Thermoreceptors Sensory Cells block 6 is decreased. The deviation signal e of block 2 is increased because reference signal r from the positive input is greater than feedback signal f from the negative input. The Human Brain regulator block 3, output signal g is changed gradually and continuously. The output signal g change rate, determined by the time constant of integration, depends only on the difference of signals r and f. This signal g is inputted into Heat Generation System of man's body block 4 that increases output t of man's body temperature according to the integration signal g of the Human Brain regulator block 3 gradually and continually. The temperature t is increased until the feedback signal f is equal to the reference signal r and deviation signal e is equal to zero. The body temperature t comes to a new equilibrium state, for example 38.4° C. The human immune system responds to Virus signal d by increasing body's temperature t and reaction to the virus signal m, to protect its host from infection, inhibit viruses' growth, and create an environment that helps kill viruses or weaken virus's ability to penetrate and replicate inside living cells.

In a simplified representation of the disturbance source Virus block 8, a more severe virus infection in the upper respiratory and GI tracts, more significantly disturbs the temperature balance. Disturbance temperature signal t1 is increased more. The output signal t2 of the summing unit block 9 is decreased accordingly. The feedback signal f of the Thermoreceptors Sensory Cells, block 6 is decreased. In turn, deviation signal e is increased and Human Brain regulator block 3, output signal g is changed gradually and continuously, but more rapidly due to a greater difference between signals r and f. The temperature t is increased more rapidly until the feedback signal f is equal to the reference signal r and deviation signal e is equal to zero. The body temperature t comes to a new equilibrium state, for example at 39.5° C. The immune system counteracts the Virus signal d even more by signal m to protect its host from infection, inhibit viruses' growth, and help kill viruses or weaken virus's ability to penetrate and replicate inside living cells.

### DETAILED DESCRIPTION OF THE INVENTION

A method of treating and/or preventing viral infections caused by common cold, influenza, and coronaviruses with or without symptoms is based on a fact that virus's survival time depends on the temperature and relative humidity, and an assumption based on scientific tests that a virus can be inactivated/killed or its ability to penetrate and replicate copies inside living cells can be weakened in high temperature environment. Creating such environment in the upper respiratory and GI tracts and preventing spread of the virus into the lower part of the respiratory tract using continuous and prolonged hot water exposure on the virus, prevents development of pneumonia and expedites recovery.

An agent delivering heat is hot, potable, boiled water the simplest, cheapest and natural product for a human. The method further reduces the risk of pneumonia by washing viruses away from the respiratory tract to GI tract. At the same time the hot water prevents development of a viral infection in the GI and urinary tracts.

The present method prevents or reduces the spread of a virus into the lower respiratory tract and prevents development of pneumonia most effectively at the early stages of an illness, when an infection just starts to develop in the upper respiratory and GI tracts, when first signs of symptoms appear, or when an individual has been tested positive for a virus, but is asymptomatic. The virus can be killed or its ability to penetrate and replicate copies inside living cells can be weakened, using continuous and prolonged hot water exposure on the virus.

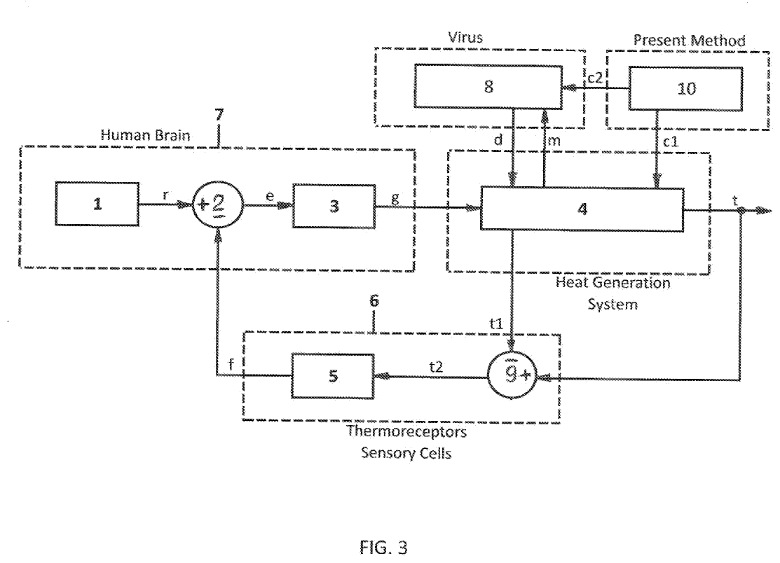

### DESCRIPTION OF THE FIG. 3

FIG. 3 is illustrated by FIG. 2 and usage of the present invention as a disturbance compensator into block diagram of feedback control system with a conventional integral controller and external source of disturbance. The disturbance compensator block 10 generates a disturbance compensator signal c1 that is inputted into controlled system block 4 and disturbance compensator signal c2 that is inputted directly into external source of disturbance block 8.

Model of a body temperature regulation with a virus disturbance and the present method

FIG. 3 illustrates a simplified model of a human body temperature regulation system under influence of a virus as a foreign body and usage of the present invention, similar to a block diagram of feedback control system with a conventional integral controller, external source of disturbance and disturbance compensator.

In this model of human body temperature regulation system under influence of a virus as a foreign body, the disturbance compensator block 10 represents Present Method of treating and preventing viral infection.

The man's body temperature t is increased in response to prolonged continuous uninterrupted drinking of hot, potable, boiled water. Temperature t is increased for two reasons:

1. The temperature is increased especially in the upper respiratory and GI tracts, in the place of infection and in place of injection (in this case, body temperature is measured in the mouth) because the water is hot and adds heat energy, improves metabolic process and blood circulation. The Present Method represented by the Disturbance Compensator block 10 generates output signal c1. This signal c1 compensates Virus signal d by entering directly into the Heat Generation System block 4, reducing temperature disturbance signal t1.

2. The continuous and prolonged drinking of hot water reduces the number and virulence of the viruses. The Present Method represented by the disturbance compensator block 10, generates output signal c2. This signal c2 unloads signal m by adding heat energy from the outside. The objective of the present method is to kill as many viruses as possible and/or weaken its ability to penetrate and replicate copies inside living cells and to help the immune system.

The number of viruses that disturb temperature balance of the human body is reduced. The body temperature t is increased and temperature's disturbance signal t1 is decreased. The summing temperature signal t2 is increased. The feedback signal f of the Thermoreceptors Sensory Cells is increased accordingly. The deviation signal e of the block 2 is decreased because feedback signal f from the negative input is increased. The regulator's output signal g of the Human Brain block 3 is decreased gradually and continuously with the change rate determined by the time constant of integration, and is dependent only on the difference of signals r and f. This signal g is inputted into the Heat Generation System of man's body block 4 that decreases man's body temperature output t, restoring the state of steady conditions for optimal functioning of the organism. The Present Method's, disturbance compensator block 10 helps the immune system to counteract the influence of the Virus block 8 by killing viruses and/or weakening its ability to penetrate and replicate copies inside living cell of organism. Immune system consumes a lot of energy to fight viruses. Present Method's block 10 unloads the immune system signal m in the fight against the Virus block 8 and takes some of the energy consumption on itself.

The temperature t is decreased until the feedback signal f is equal to the reference signal r and deviation signal e is equal to zero. The body temperature t comes to a new equilibrium state, for example back to 37.4° C. from 39.5° C.

The longer hot water is consumed and the higher personal maximal temperature the more viruses can be killed or weakened, the greater the possibility that body temperature t returns to normal and human body temperature regulation system returns back to the conventional block diagram described by FIG. 1. The body temperature t is a good indicator of the severity of the disease and the correctness of the application of the present method.

In order to make treatment and/or prevention viral infections according to the invention particularly effective, all the detail instructions are implemented below.

### INSTRUCTIONS FOR THE USE OF THIS METHOD

As mention above the present method uses regular potable water and involves the use of boiled water. This is a very important feature because large volume of consumption of water requires that water must be without or with minimum amount of viruses, bacteria, and parasites. The water is boiled by regular teapots, coffee machines, heating elements or over open fire.

It is known that coffee and tea beverages are brewed at temperatures that are sufficiently high to cause immediate and serious risk for scald injuries. On the other hand, numerous research studies have been performed to identify the preferred consumption temperatures for hot beverages. The outcome of these mutually reinforcing studies is that the preferred drinking temperatures are significantly below the often-encountered brewing temperatures. Consequently, there is great need to distinguish brewing temperatures from serving temperatures. A more rationale recommended range of service temperatures in USA is 55° C. to 71 ° C. This recommendation balances a range of consumer preferences and safety. So the temperature of water 55° C. is safe hot water temperature that can be consumed from cup/mug by a person. The best cup/mug that can be used for present method is double wall vacuum Insulated technology to prevent heat transfer to the outer walls of the tumbler, keeping drinks hot.

The parameters specified in the claims of the invention cover healthy adult population living in temperate climate with weight ranging from 50 kg to 100 kg. For people with underlying health problems all these limitations are defined by a doctor and can be changed in accordance with patient's health conditions. All instructions below are implemented for healthy adults with average weight of 70 kg.

For each person maximum drinking temperature of water, which is acceptable, is identified as personal maximal temperature of water (PMTW). The hot water is consumed at a personal maximal temperature of water that a person can drink. A procedure of determination the personal maximal temperature of water is offered at present invention, where temperature is determined by volume of small sips of water within 4-8 mL or 0.8-1.6 teaspoons that is used for dosing of medicine, to prevent scald burns injury, allow further consumption, limit significant increase consumption rate, and keep it within specified range.

The procedure for determination of personal maximal temperature of water comprises first steps of drinking at least 2 sips with said minimum water temperature in a cup/mug of 55° C.: if the sips are within volume range of said small sip and cannot be more than small sips, the personal maximal temperature of water is 55° C., if the sips can be more than said small sips, the water is not hot enough and is diluted with hotter water; repeating the first steps with hotter water until temperature of water allows to proceed to second steps of drinking at least 5 said small sips of water within selected small sip's volume range only, this temperature is personal maximal temperature of water that can be measured by regular instant read beverage thermometer. The individual ability of usage more volume sips indicates that the water temperature is not personal maximal temperature and the need for addition of hotter water.

The present method comprises further water consumption procedure with selected personal maximal temperature of water comprising drinking the rest of the cup/mug of water and the next filled cups/mugs of water by sips within selected volume range of small sips 4-8 mL or approximately 6 mL±30%. The duration of time for one cycle of small sips (between starts of two consecutive sips) with volume of 6 mL (in the middle) is 6 ml×60 sec/37 mL≈10 seconds, with the consumption rate of 37 mL/min (in the middle) that is calculated from: (15 mL/min/(100 kg-50 kg)×70 kg+16 mL/min)±1 mL/min=36-38 mL/min. Assume that the swallowing of small sip of hot water with personal maximal temperature of water takes approximately 2 seconds then the interval between sips equals 8 seconds. So, the interval range between the two consecutive small sips of hot water is 8 sec ±30% or approximately 6-10 seconds for people with weight of 70 kg.

As proved by scientific research, the survival time of a virus depends on the temperature of the environment in which it is located. According to the claims, the duration of hot water consumption depends on the personal maximal temperature of water—the higher the PMTW, the shorter the duration of water consumption. The general formula for the minimum duration of hot water consumption is 292.5 min×(75° C.-55° C.)/PMTW° C. As an example, all instructions below are implemented for personal maximal temperature of water PMTW=65° C., in the middle of the temperature range from 55° C. to 75 ° C., used for serving hot beverages around the world. In this case the minimum duration of hot water consumption is 292.5 min×(75° C.-55° C.)/65° C.=90 min and the maximum duration of hot water consumption is 292.5 min×20° C. /65° C.+60 min=150 min.

During continuous, uninterrupted 90 minutes of treatment of viral infection with symptoms the individual should drink hot water at personal maximal temperature of water, with the consumption rate 36-38 mL/min. Minimum volume of consumed water will be ((15 mL/min/(100 kg-50 kg)×70 kg+16 mL/min)±1 mL/min)×90 min=3300 mL. If after 90-minute minimum duration of water consumption and minimum water volume consumption of 3300 mL the symptoms have disappeared or have reduced significantly and person feels better based on subjective evaluation, the treatment process should be stopped. The specified minimum duration 90 minutes and minimum consumption volume 3300 mL, as well as maximum duration 150 minutes and maximum consumption volume ((15 mL/min/(100 kg-50 kg)×70 kg+16 mL/min)±1 mL/min)×150 min≈5500 mL are requirements for the person with 70 kg weight.

For example: If you start treating yourself immediately after first signs of illness, you can drink at the rate of 36-38 mL/min for 90 minutes for a total volume of 3300 mL of hot water, but not less. If the patient no longer experiences symptoms, it is not necessary to drink more than 3300 mL of water. If you continue to experience symptoms you should drink more than 3300 mL of water. The symptoms are indicators of an illness. Without symptoms you don't know that you are sick. Even with symptoms, you don't know if it is cold, influenza, or severe acute respiratory syndrome coronavirus. Each patient experiences and evaluates symptoms uniquely and subjectively, even common symptoms. However, in this method the subjectivity, regardless of the symptoms, is limited by the requirement to consume a minimum of 3300 mL of hot water and a maximum of 5500 mL of hot water for person with weight 70 kg. In between 3300 mL and 5500 mL the consumption volume is determined by symptoms. If after consumed 3300 mL of hot water the patient still feels the same symptoms as before the treatment, the patient should continue treatment by this method until an individual feels the symptoms have disappeared or have been significantly reduced by patient's subjective evaluation, but not to exceed the maximum volume consumption of 5500 mL. The symptoms are used as an indicator of the end of the treatment process by present method for each person. The uniqueness in the application of the present method is that the temperature, time, and consumption's volume are selected and controlled by an individual for healthy adults or by a doctor, in cases where patient's underlying health conditions require consultation with a doctor. Only the individual can modify the metes and bounds of duration (within range of 90-150 minutes), water volume consumption (within 3300-5500 mL range) with selected water volume consumption rate within 36-38 mL range and-personal maximal temperature of water 65° C. Sometimes the symptoms relief occurs earlier than inactivation of viruses in the upper respiratory and GI tracts, but sometimes later. For example: fever can start to drop after 1 hour of the treatment, whereas muscle pain may go away after 2 hours of treatment. In any instances individual with weight 70 kg and personal maximal temperature of water 65° C. should drink hot water minimum 90 minutes and minimum 3300 mL and not exceed maximum 150 minutes and 5500 mL.

### PRACTICAL USE OF THE INVENTION

### Treatment

This method is the most effective for treatment during the early stages of an infection, when first signs of symptoms appear. To simplify practical use, the minimum 90 minutes and maximum 150 minutes are selected as basic durations of hot water consumption because it covers (100±15) % and (100±10) % respectively for all possible numbers of minimum and maximum durations of hot water consumption within selected personal maximal temperature of water range from 55° C. to 75° C. If individual with 70 kg weight experiences symptoms such as fever, sore throat, cough, shortness of breath, muscle pain and so on, he/she should begin following the presented method immediately.

### Treatment Procedure:

1. Boil regular potable water and pour into a cup/mug (preferable with double walls) and allow cooling or dilute with cold water to the temperature, when it can be safely consumed, based on an individual's ability to drink hot water, but not less than 55° C.
2. Start drinking the hot water by sips. If sip's volume can be more than volume of 2 regular teaspoons then temperature of water at this point is too low and need addition of hotter water. Repeat addition of hotter water until temperature of water in cup/mug allows sip's volume to be only less than volume of 2 regular teaspoons. This temperature is personal maximal temperature of water that individual can drink.
3. Water should be consumed at a constant, steady pace with interval between sips approximately 6-10 seconds.
4. Once the first cup is finished, continue step 3, with personal maximal temperature of water determined in steps 1, 2 with minimal breaks between cups/mugs, to ensure a continuous steady pace of consumption.

Continue with step 4 and end the treatment process when:
a. Duration of hot water consumption is not less than 90 minutes and hot water consumption volume is not less than 3300 mL, if individual do not experience any symptoms thereafter.
b. Duration of hot water consumption is more than 90 minutes and consumption volume is more than 3300 mL, if individual continue feels the symptoms experienced prior to applying this method; hot water is consumed until the symptoms have gone away or have been significantly reduced by person's subjective evaluation.
c. Duration of hot water consumption is not more than 150 minutes and hot water consumption volume is not more than 5500 mL, if the symptoms experience prior to applying this method is continued.

An individual should expect some or significant amount of sweating throughout the process.

### Prevention

The method is used for treatment of viral infection without symptoms when an individual doesn't know when he/she was contaminated but has tested positive for a virus, and/or for prevention when an individual believes they were in a contaminated environment (e.g., around sick people). Prevention method should be utilized in a similar manner as other proactive steps identified by medical professionals, such as washing your hands, using hand sanitizer or wearing a facemask. This method could be used more frequently during virus seasons. It is a good practice to follow on a consistent basis.

### Prevention Procedure:

Repeat with steps 1 through 4 of the above described Treatment Procedure, but with the duration of hot water consumption range is from 30 minutes to 60 minutes and the amount of consumed hot water range is from 1100 mL to 2200 mL (for person with weight 70 kg).

Depending on an individual's internal condition, overall medical and health condition, as well as external environment, the treatment and/or prevention procedures can be used in different combinations, with different frequency, within a day, days, weeks or months. This method does not treat bronchitis or pneumonia. It is the most effective at the early stage of illness. It can expedite recovery and prevent complications.

The present invention can be used as a stand-alone method of treatment and prevention viral infection as well as additional to all existing conventional methods.

This method is safe, and there are no side effects. However, people with special health conditions have to use this method with permission of their physician or under physician's control. It is a personal responsibility of each individual to use this method appropriately and based on the user's physical and health condition, without causing harm to self.

Time frame of use of the present method

### EXAMPLE 1:

First day:
1. 11.00 AM. An individual takes their temperature for the first time because he/she feels sick.

Temperature is 39.5° C. (103.1° F.).
2. 11.15 AM. Individual starts to drink hot boiled water according to the Treatment Procedure described above.
3. 6.00 PM. Individual takes their temperature again. If temperature is significantly less than 39.5° C. (103.1° F.), for example 37.4° C. (99.3° F.) no further actions is required pertaining to the method on this day. If temperature is more than 38° C. (100.4° F.) the individual may use the Prevention Procedure described above after 6 PM on the same day.

Second day:
4. If by 12 PM temperature has not increased, treatment by this method is complete.
5. If by 12 PM temperature has increased significantly, repeat the Prevention Procedure again, one more time.

After using the present method for two days, as presented in the examples above, the usage of this method is finished. Furthermore, in all circumstances, the conventional methods of treatment, including medical assistance, should be considered and followed appropriately.

### EXAMPLE 2:

First day:
1. 5.00 PM. An individual takes their temperature for the first time because he/she feels sick. Temperature is 40.5° C. (105° F.).
2. 5.15 PM. Individual starts to drink hot, boiled water according to the Treatment Procedure described above. At the same time the individual may take fever reducer.
3. 10.00 PM. Individual takes their temperature again. If temperature is significantly less than 40.5° C. (105° F.), for example 37.4° C. (99.3° F.) no further action is required pertaining to the method, on this day. If temperature is more than 38° C. (100.4° F.), the individual may take fever reducer or multi symptoms relief medicine and go to bed.

Second day:
4. If by 12 PM temperature has not increased, treatment by this method is complete.
5. If by 12 PM temperature has increased significantly, repeat the Treatment Procedure again, one more time.

The usage of this method is finished in any circumstances. If temperature is still high, after using the method, as presented in the examples above, the conventional methods of treatment, including medical assistance, should be considered and followed appropriately.

### Testing

### EXAMPLE 1:

This method was tested late December 2012 by the author of this invention for treatment of flu. During the onset of slight symptoms of sore throat and muscle pain in the legs, the individual started to follow this method's Treatment Procedure immediately as described above. The individual achieved full recovery, with none of the original symptoms present, within one day.

### EXAMPLE 2:

This method was tested late February 2020 by the author of this invention for treating presumably coronavirus COVID-19. During the onset of symptom of high fever 40.5° C., the individual started to follow this method's Treatment Procedure immediately as described above. The individual's fever was gone within one day. The individual was isolated for the next 6 days to prevent infecting other people. The individual continued to experience residual light cough, lasting 4 weeks. After the first two weeks of the cough, the individual visited a doctor and took a chest X-rays to confirm that the lungs were clear and that the infection did not develop into pneumonia.

The author of this invention personally used this method on himself, members of family, and friends with fever, muscle pain, weakness, running nose, sore throat, and cough. In each case, most of the symptoms disappeared or significantly reduced, based on subjective evaluation after one or two days of treatment. All specified metes and bounds were obtained empirically over 10 years of the author experience, which have tested for cold, flu, and presumably Covid-19. Every individual who has used this method has recovered expeditiously and without any complications.

### CONCLUSIONS

Substantial benefits of the proposed method of treatment and prevention of viral infections are obvious. Correct and timely application of the treatment and prevention procedures shows the high efficacy of the proposed method. This method is the most readily available, cheapest, safest, and easiest method of treatment and prevention of viral infections. This means that the method can help slow down the spread of the cold, flu, coronaviruses epidemics or pandemics. Recognition of this method by the scientific community can make this method one of the most popular treatment and prevention option to manage viral infections around the world. Meanwhile, the study of the hot water effect on viruses as a new scientific method for treatment and prevention of viral infection should be carried out in full capacity including comprehensive clinical and statistical research. Today, creation of vaccines is the only way to fight viruses. A better understanding of the influence of temperature on a virus inside human body and the application of this method is also needed to guide future efforts in virus infection treatment and prevention.

What is claimed is:

1. A method of treating and/or preventing viral infections caused by common cold, influenza, and coronaviruses, whose survival time depends on temperature, with symptoms or without symptoms, comprises prolonged, continuous, uninterrupted heat exposure to the virus in the upper respiratory and gastrointestinal (GI) tracts creating high temperature environment by consuming hot, potable, boiled water.

2. The method of claim 1, wherein a selected minimum temperature of said hot water is 55° C.

3. The method of claim 2, wherein said continuous, uninterrupted heat exposure to the virus, selected hot water consumption rate is (15 mL/min/50 kg×(user weight in kg)+16 mL/min)±1 mL/min tolerance for selected user's weight range from 50 kg to 100 kg.

4. The method of claim 3, wherein said hot, potable, boiled water is consumed by small sips.

5. The method of claim 4, wherein selected volume range of said small sips is 4-8 mL.

6. The method of claim 5, wherein the hot water is consumed by individual at a personal maximal temperature of water (PMTW) that a person can drink.

7. The method of claim 6 further comprises determination of said personal maximal temperature of water, wherein temperature of water allows drinking of hot water only by said small sips, whereas ability to drink hot water with larger volume sip indicates that temperature of water is not personal maximal temperature of water and the need for addition of hotter water.

8. The method of claim 7, wherein said method of treating viral infection with symptoms comprises prolonged heat exposure to the virus with said personal maximal temperature of water, minimum duration of hot water consumption is (292.5 min×20° C/ PMTW° C.) minutes, and maximum duration of hot water consumption is (292.5 min×20° C./PMTW° C.+60 min) minutes for selected hot water temperature range from 5° C. to 75 ° C.

9. The method of claim 8, wherein said method of treating viral infections with symptoms, within specified said minimum and maximum duration of hot water consumption, the symptoms' disappearance or subjective evaluation of symptoms' reduction are used as an indicator of the end of the treatment process by said method for each person.

10. The method of claim 7, wherein said the method of treating and/or preventing viral infection without symptoms comprises prolonged heat exposure to a virus, minimum duration of hot water consumption is 30 minutes, and maximum duration of hot water consumption is 60 minutes.
